(19) United States
(12) Patent Application Publication
(10) Pub. No.: US 2025/0281073 A1
(43) Pub. Date: Sep. 11, 2025

(54) Chemical Detection Using Single-Photon Avalanche Diodes

(71) Applicant: Artilux, Inc., Menlo Park, CA (US)

(72) Inventors: Neil Y. NA, San Jose, CA (US); Yi-Chuan TENG, HSINCHU COUNTY (TW)

(21) Appl. No.: 19/052,086

(22) Filed: Feb. 12, 2025

Related U.S. Application Data
(60) Provisional application No. 63/562,263, filed on Mar. 7, 2024.
(60) Provisional application No. 63/685,724, filed on Aug. 22, 2024.
(60) Provisional application No. 63/688,311, filed on Aug. 29, 2024.

(51) Int. Cl. A61B 5/145; A61B 5/00; A61B 5/1455

(52) U.S. Cl. CPC A61B 5/14532; A61B 5/1455; A61B 5/7264; A61B 5/725

(57) ABSTRACT

Described herein are systems and methods for non-invasive glucose detection using an optical sensing apparatus. The optical sensing apparatus comprises one or more single-photon avalanche diodes (SPADs) and one or more processors. The one or more SPADs may be configured to receive light pulses having one or more wavelengths that have interacted with biological tissues over a plurality of time cycles and convert the light pulses into a plurality of electrical signals. Each of the one or more SPADs may comprise an absorption region formed on a substrate, wherein the absorption region comprises germanium or germanium containing tin, and wherein the substrate comprises silicon. The one or more processors may be configured to identify, for each of the plurality of time cycles, particular time slots representing a time duration that each of the light pulses has interacted with glucose molecules and determine one or more characteristics of glucose molecules.

### CROSS-REFERENCE TO RELATED APPLICATIONS

This application claims priority to and the benefit of U.S. Provisional Patent Application No. 63/562,263, filed Mar. 7, 2024; U.S. Provisional Patent Application No. 63/685,724, filed Aug. 22, 2024; and U.S. Provisional Patent Application No. 63/688,311, filed Aug. 29, 2024, the contents of each of which is incorporated herein in their entirety.

### TECHNICAL FIELD

This application relates to sensors, and more particularly, to optical sensors that detect light in the near-infrared (NIR, e.g., wavelength range from 780 nm to 1000 nm, or any similar wavelength range as defined by a particular application), the shortwave infrared (SWIR, e.g., wavelength range from 1000 nm to 3000 nm, or any similar wavelength range as defined by a particular application). As a technical advantage, the photodetector described herein may be used for chemical detection (e.g., glucose) in a body in a non-invasive manner.

### BACKGROUND

Diabetes mellitus is a group of endocrine diseases characterized by an inability to regulate blood glucose levels appropriately. Failure to maintain appropriate blood glucose levels can lead to serious illness and even death. Accordingly, it is vital for individuals (e.g., diabetic individuals) to monitor their blood glucose levels throughout the day so they can treat any hypo-glycemic (low blood sugar) or hyper-glycemic (high blood sugar) states.

Conventional methods of measuring blood glucose levels involve pricking a finger to collect a small amount of blood. This can be uncomfortable and even a painful process. Another conventional method of measuring blood glucose levels is by using a conventional continuous glucose monitor. A conventional continuous glucose monitor is a wearable device typically worn on the abdomen, leg, or arm of the diabetic individual. Some conventional glucose monitors include a small sensor that is inserted subcutaneously with a needle. The needle is then removed, while the sensor remains in the body, sitting in the interstitial fluid. Although the needle may not remain in the patient's body, the insertion, wearing, and removal process can also be uncomfortable and painful, or even cause bleeding or infection.

Accordingly, there is a desire for chemical detection (e.g., glucose monitoring) that is non-invasive, and allows users to monitor their chemical (e.g., blood glucose) levels as accurately as needed.

### SUMMARY

The techniques described herein address the issues of conventional blood glucose detection by providing a non-invasive glucose monitor based on single-photon avalanche diodes (SPAD). The non-invasive glucose monitor disclosed herein may use SPAD photodetectors to detect chemicals (such as glucose) using time-resolved spectroscopy. SPAD photodetectors may provide time-resolved information related to the tissues with direct time-of-flight (dToF) measurements.

The spectroscopy may involve using light sources in near-infrared (NIR) and/or short-wave infrared (SWIR) ranges. For example, the light sources may be dual-SWIR-wavelength light sources. The dual-SWIR-wavelength light sources and associated sensors may be specifically selected for detecting one or more target chemicals. In some examples, to enhance the performance of signal calibration, more than two wavelengths may be used. Further, using a germanium-silicon (GeSi) SPAD array and/or on-chip bandpass filter and/or temporally interchanged illumination, the non-invasive glucose monitor may be able to collect signals at each wavelength individually.

By using SWIR dToF technology, the disclosed systems and methods may be fast, reliable, non-invasive, cost-effective, and efficient in collecting time-domain photon counting signals. By utilizing the time-domain photon counting signals, the disclosed systems and methods may be able to obtain target chemical information (e.g., glucose concentrations) directly without using computationally heavy data processing or big data-driven algorithms.

While the present disclosure refers to determining glucose levels, it is understood that the systems and methods described herein can be used to determine and monitor other target analytes as long as the target analyte can be identified through appropriately chosen wavelengths. In some examples, the systems and methods disclosed herein can detect target chemical information (e.g., glucose levels).

An optical sensing apparatus for glucose detection is disclosed. The optical sensing apparatus comprises: one or more single-photon avalanche diodes configured to: receive light pulses having one or more wavelengths that have interacted with biological tissues over a plurality of time cycles; and convert the light pulses into a plurality of electrical signals, wherein each of the one or more single-photon avalanche diodes comprises: an absorption region formed on a substrate, wherein the absorption region comprises germanium or germanium containing tin, and wherein the substrate comprises silicon; circuitry configured to process the plurality of electrical signals to generate a plurality of digital electrical signals; one or more processors configured to: identify, for each of the plurality of time cycles, particular time slots representing a time duration that each of the light pulses has interacted with glucose molecules; and determine, based on a subset of the plurality of digital electrical signals that correspond to the particular time slots, one or more characteristics of glucose molecules associated with the biological tissues.

Additionally or alternatively, in some embodiments, the one or more single-photon avalanche diodes are arranged in a one-dimensional array or a two-dimensional array.

Additionally or alternatively, in some embodiments, determining the one or more characteristics of the glucose molecules associated with the biological tissues further comprises determining, based on a subset of the plurality of digital electrical signals that correspond to specific pixel locations in the one-dimensional array or the two-dimensional array, the one or more characteristics of glucose molecules associated with the biological tissues.

Additionally or alternatively, in some embodiments, the one or more single-photon avalanche diodes comprise one or more wavelength filters arranged on the absorption regions for passing the one or more wavelengths of the light pulses.

Additionally or alternatively, in some embodiments, each of the particular time slots is determined based on a timing jitter associated with the one or more single-photon avalanche diodes.

Additionally or alternatively, in some embodiments, the circuitry comprises a time-to-digital converter.

Additionally or alternatively, in some embodiments, identifying the particular time slots further comprises identifying the particular time slots based on one or more of an effective index associated with the biological tissues, an absorption coefficient associated with the biological tissues, or a scattering coefficient associated with the biological tissues.

Additionally or alternatively, in some embodiments, identifying the particular time slots further comprises identifying the particular time slots based on determining that the light pulses have transmitted through the biological tissues.

Additionally or alternatively, in some embodiments, identifying the particular time slots further comprises identifying the particular time slots based on determining that the light pulses have reflected from the biological tissues.

Additionally or alternatively, in some embodiments, determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

Additionally or alternatively, in some embodiments, the light pulses have wavelengths in two or more of (i) a range between 1000 nm to 1400 nm, (ii) a range between 1500 nm to 1700 nm, (iii) a range between 1800 nm to 1900 nm, or (iv) a range between 2000 nm to 2250 nm.

A method for detecting glucose in biological tissues using an optical sensing apparatus is disclosed. The method comprises: receiving, by one or more single-photon avalanche diodes, light pulses having one or more wavelengths that have interacted with the biological tissues over a plurality of time cycles; converting, by the one or more single-photon avalanche diodes, the light pulses into a plurality of electrical signals, wherein each of the one or more single-photon avalanche diodes comprises an absorption region formed on a substrate, wherein the absorption region comprises germanium or germanium containing tin, and wherein the substrate comprises silicon; processing, by circuitry, the plurality of electrical signals to generate a plurality of digital electrical signals; identifying, by one or more processors and for each of the plurality of time cycles, particular time slots representing a time duration that each of the light pulses has interacted with glucose molecules; and determining, by the one or more processors and based on a subset of the plurality of digital electrical signals that correspond to the particular time slots, one or more characteristics of glucose molecules associated with the biological tissues.

Additionally or alternatively, in some embodiments, the one or more single-photon avalanche diodes are arranged in a one-dimensional array or a two-dimensional array.

Additionally or alternatively, in some embodiments, determining the one or more characteristics of the glucose molecules associated with the biological tissues further comprises determining, based on a subset of the plurality of digital electrical signals that correspond to specific pixel locations in the one-dimensional array or the two-dimensional array, one or more characteristics of glucose molecules associated with the biological tissues.

Additionally or alternatively, in some embodiments, each of the particular time slots is determined based on a timing jitter associated with the one or more single-photon avalanche diodes.

Additionally or alternatively, in some embodiments, identifying the particular time slots further comprises identifying the particular time slots based on one or more of an effective index associated with the biological tissues, an absorption coefficient associated with the biological tissues, or a scattering coefficient associated with the biological tissues.

Additionally or alternatively, in some embodiments, identifying the particular time slots further comprises identifying the particular time slots based on determining that the light pulses have transmitted through the biological tissues.

Additionally or alternatively, in some embodiments, identifying the particular time slots further comprises identifying the particular time slots based on determining that the light pulses have reflected from the biological tissues.

Additionally or alternatively, in some embodiments, determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

An optical sensing apparatus for glucose detection is disclosed. The optical sensing apparatus comprises: one or more single-photon avalanche diodes configured to: receive light pulses that have interacted with biological tissues over a plurality of time cycles; and convert the light pulses into a plurality of electrical signals, wherein each of the one or more single-photon avalanche diodes comprises an absorption region formed on a substrate, wherein the absorption region comprises germanium or germanium containing tin, and wherein the substrate comprises silicon; circuitry configured to process the plurality of electrical signals to generate a plurality of digital electrical signals; and one or more processors configured to: identify, for each of the plurality of time cycles, particular time slots representing a time duration that each of the light pulses has interacted with glucose molecules; and determine, using a machine-learning model and based on a subset of the plurality of digital electrical signals that correspond to the particular time slots, one or more characteristics of glucose molecules associated with the biological tissues.

An optical sensing apparatus for glucose detection is disclosed. The optical sensing apparatus comprises: one or more single-photon avalanche diodes configured to: receive light pulses having at least two wavelengths that have interacted with biological tissues over a plurality of time cycles, wherein the at least two wavelengths include a signal wavelength and a reference wavelength; and convert the light pulses into a plurality of electrical signals, wherein each of the one or more single-photon avalanche diodes comprises an absorption region formed on a substrate, wherein the absorption region comprises germanium or germanium containing tin, and wherein the substrate comprises silicon; and one or more processors configured to: determine, based on the plurality of electrical signals, a time duration that each of the light pulses has interacted with glucose molecules; and determine, based on one or more of (i) the time duration, (ii) one or more characteristics associated with interactions between the signal wavelength and glucose molecules, (iii) one or more characteristics associated with interactions between the reference wavelength and glucose molecules, (iv) input intensities of the light pulses, or (v) output intensities of the light pulses, one or more characteristics of glucose molecules associated with the biological tissues.

Additionally or alternatively, in some embodiments, the one or more single-photon avalanche diodes are arranged in a one-dimensional array or a two-dimensional array.

Additionally or alternatively, in some embodiments, the one or more single-photon avalanche diodes comprise one or more wavelength filters arranged on the absorption regions for passing the signal wavelength and the reference wavelength.

Additionally or alternatively, in some embodiments, determining the time duration that each of the light pulses has interacted with glucose molecules further comprises identifying, for each of the plurality of time cycles, particular time slots representing the time duration that each of the light pulses has interacted with glucose molecules.

Additionally or alternatively, in some embodiments, identifying the particular time slots representing the time duration that each of the light pulses has interacted with glucose molecules further comprises identifying the particular time slots based on a histogram comparison between the light pulses at the signal wavelength and the light pulses at the reference wavelength.

Additionally or alternatively, in some embodiments, determining the time duration that each of the light pulses has interacted with glucose molecules further comprises determining an average time duration over multiple time durations that the light pulses have interacted with glucose molecules.

Additionally or alternatively, in some embodiments, the one or more characteristics associated with interactions between the signal wavelength and glucose molecules comprises a skin absorption coefficient of the signal wavelength, a skin scattering coefficient of the signal wavelength, and a first effective index associated with the biological tissues at the signal wavelength, and the one or more characteristics associated with interactions between the reference wavelength and glucose molecules comprises a skin absorption coefficient of the reference wavelength, a skin scattering coefficient of the reference wavelength, and a second effective index associated with the biological tissues at the reference wavelength.

Additionally or alternatively, in some embodiments, the first effective index associated with the biological tissues at the signal wavelength is appropriately equal to the second effective index associated with the biological tissues at the reference wavelength.

Additionally or alternatively, in some embodiments, the one or more characteristics of glucose molecules associated with the biological tissues comprise a concentration of the glucose molecules associated with the biological tissues.

Additionally or alternatively, in some embodiments, determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

Additionally or alternatively, in some embodiments, determining the one or more characteristics of the glucose molecules associated with the biological tissues further comprises determining the one or more characteristics of the glucose molecules using a neural network model, wherein input data for the neural network model includes user characteristics that affect how the light pulses interact with the biological tissues, and wherein the user characteristics include one or more of skin color, gender, or age information.

Additionally or alternatively, in some embodiments, the reference wavelength and the signal wavelength are in a range between 1000 nm to 1400 nm.

A method for detecting glucose in biological tissues using an optical sensing apparatus having one or more single-photon avalanche diodes is disclosed. The method comprises: receiving, by the one or more single-photon avalanche diodes, light pulses having at least two wavelengths that have interacted with biological tissues over a plurality of time cycles, wherein the at least two wavelengths include a signal wavelength and a reference wavelength; converting, by the one or more single-photon avalanche diodes, the light pulses into a plurality of electrical signals, wherein each of the one or more single-photon avalanche diodes comprises an absorption region formed on a substrate, wherein the absorption region comprises germanium or germanium containing tin, and wherein the substrate comprises silicon; determining, based on the plurality of electrical signals, a time duration that each of the light pulses has interacted with glucose molecules; and determining, based on one or more of (i) the time duration, (ii) one or more characteristics associated with interactions between the signal wavelength and glucose molecules, (iii) one or more characteristics associated with interactions between the reference wavelength and glucose molecules, (iv) input intensities of the light pulses, or (v) output intensities of the light pulses, one or more characteristics of glucose molecules associated with the biological tissues.

Additionally or alternatively, in some embodiments, determining the time duration that each of the light pulses has interacted with glucose molecules further comprises determining an average time duration over multiple time durations that the light pulses have interacted with glucose molecules.

Additionally or alternatively, in some embodiments, determining the time duration that each of the light pulses has interacted with glucose molecules further comprises identifying, for each of the plurality of time cycles, particular time slots representing the time duration that each of the light pulses has interacted with glucose molecules.

Additionally or alternatively, in some embodiments, identifying the particular time slots representing the time duration that each of the light pulses has interacted with glucose molecules further comprises identifying the particular time slots based on a histogram comparison between the light pulses at the signal wavelength and the light pulses at the reference wavelength.

Additionally or alternatively, in some embodiments, the one or more characteristics associated with interactions between the signal wavelength and glucose molecules comprises a skin absorption coefficient of the signal wavelength, a skin scattering coefficient of the signal wavelength, and a first effective index associated with the biological tissues at the signal wavelength, and the one or more characteristics associated with interactions between the reference wavelength and glucose molecules comprises a skin absorption coefficient of the reference wavelength, a skin scattering coefficient of the reference wavelength, and a second effective index associated with the biological tissues at the reference wavelength.

Additionally or alternatively, in some embodiments, the first effective index associated with the biological tissues at the signal wavelength is appropriately equal to the second effective index associated with the biological tissues at the reference wavelength.

Additionally or alternatively, in some embodiments, the one or more characteristics of glucose molecules associated with the biological tissues comprise a concentration of the glucose molecules associated with the biological tissues.

Additionally or alternatively, in some embodiments, determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

Additionally or alternatively, in some embodiments, determining the one or more characteristics of the glucose molecules associated with the biological tissues further comprises determining the one or more characteristics of the glucose molecules using a neural network model, and wherein input data for the neural network model includes user characteristics that affect how the light pulses interact with the biological tissues, and wherein the user characteristics include one or more of skin color, gender, or age information.

Additionally or alternatively, in some embodiments, the reference wavelength and the signal wavelength are in a range between 1000 nm to 1400 nm.

It will be appreciated that any of the variations, aspects, features, and options described in view of the systems apply equally to the methods and vice versa. It will also be clear that any one of the above variations, aspects, features, and options can be combined.

### BRIEF DESCRIPTION OF THE DRAWINGS

Aspects of the disclosure are described, by way of example only, with reference to the accompanying drawings. The foregoing aspects and many of the advantages of this application will become more readily appreciated as the same becomes better understood by reference to the following detailed description, when taken in conjunction with the accompanying drawings:

FIG. 1 illustrates an example system for chemical detection, according to some embodiments.

FIG. 2 illustrates an example relationship graph between wavelengths and absorption coefficients, according to some embodiments.

FIG. 3 illustrates a representative cross-sectional view of biological tissues, according to some embodiments.

FIG. 4A illustrates a cross-sectional view of an example photodetector, according to some embodiments.

FIG. 4B illustrates a top view of an example photodetector, according to some embodiments.

FIG. 5A illustrates a cross-sectional view of an example photodetector, according to some embodiments.

FIG. 5B illustrates a top view of an example photodetector, according to some embodiments.

FIG. 6 illustrates an example neural network model, according to some embodiments.

FIG. 7 illustrates an example flow for detecting glucose, according to some embodiments.

FIGS. 8A and 8B illustrate an example system for chemical detection, according to some embodiments.

FIG. 9A illustrates an example histogram, according to some embodiments.

FIG. 9B illustrates an example of selecting a reference wavelength to stabilize reference light intensity, according to some embodiments.

FIG. 10 illustrates an example relationship between optical measurements and chemical concentration, according to some embodiments.

FIG. 11 illustrates an example flow for detecting glucose, according to some embodiments.

Like reference numbers and designations in the various drawings indicate like elements.

### DETAILED DESCRIPTION

Reference will now be made in detail to implementations and various aspects and variations of systems and methods described herein. Although several example variations of the systems and methods are described herein, other variations of the systems and methods may include aspects of the systems and methods described herein combined in any suitable manner having combinations of all or some of the aspects described.

As described, treatment of diabetes requires monitoring of a diabetic individual's blood glucose levels. Conventional methods of monitoring blood glucose levels are invasive and may cause inconvenience and pain to the individual. Accordingly, disclosed herein are systems and methods for non-invasively detecting blood glucose concentration with time-resolved spectroscopy using SPAD photodetectors (e.g., GeSi SPAD photodetectors, or other SPAD photodetectors formed using another material or material compound).

Spectroscopy is the scientific field that studies the interaction between matter and electromagnetic radiation, focusing on how light is scattered, absorbed, and emitted by different materials. Spectroscopy may involve emitting light at particular wavelengths and directing the light at a substance. Light that passes through the substance may be detected by one or more photodetectors. Absorption spectroscopy is a type of spectroscopy that may measure a loss of electromagnetic energy after it is used to illuminate the substance. Many substances have unique and identifiable spectral characteristics. By identifying which wavelengths were absorbed by the substance, the substance may be identified. Additionally, absorption spectroscopy may be used to determine the concentration of an analyte in a substance as the amount of absorbed light is proportional to the amount of the analyte in the substance.

Time-resolved spectroscopy is a spectroscopy technique that may be used to study how a substance's property changes over time. Time-resolved spectroscopy may include steps such as exciting a sample with a short pulse of light (e.g., femtoseconds pulse width, or picoseconds pulse width, or any other pulse width that provides the suitable resolution), and detect how the sample interacts (e.g., absorbs, emits, scatters, etc.) with the light pulse over time.

Detecting target chemical information (e.g., glucose levels) in the body with light, a technique explored in non-invasive glucose monitoring, presents several challenges due to the nature of light interactions with biological tissues and the characteristics of glucose molecules. For example, glucose molecules absorb light in a similar manner to many other substances found in the human body, such as water, hemoglobin, muscle, and lipid, making it difficult to isolate the desired data. Glucose molecules and hemoglobin may both have high absorbance of light within one or more wavelength ranges. Glucose molecules and water molecules may both have high absorbance of light within one or more other wavelength ranges. Glucose molecules and lipid molecules may both have high absorbance of light within yet another one or more different wavelength ranges. Accordingly, the systems and methods disclosed herein may utilize wavelengths of light that may be selected such that glucose molecules have high absorbance, but other interfering molecules (e.g., hemoglobin, water, and/or lipids) have low absorbance.

As another example, when light passes through human tissues, it is not only absorbed but also scattered in various directions due to the heterogeneous nature of the tissues. While conventional methods of source-detector separation (SDS) based calibration may reduce the signal offset caused by light entering and exiting the tissues, it is only approximate and not accurate. Accordingly, the systems and methods described herein may utilize time-resolved techniques that reveal the behavior of light scattering and absorption in the time domain in response to the change of the concentration of glucose molecules, thereby increasing the accuracy of the measurements.

As yet another example, light in the visible and near-infrared range (NIR) has limited penetration depth through skin and tissue, which limits the ability of the light to reach the blood vessels where glucose resides, making it challenging to collect a high quality signal for accurate measurement. Accordingly, the systems and methods disclosed herein may utilize photodetectors with high sensitivity, which may enable detection of light on a single photon scale. Further, the systems and methods herein may utilize wavelengths in the short-wave infrared (SWIR) range, which may have greater penetration depth than wavelengths in the NIR range.

Conventional systems may also use SWIR single-photon detectors that require low temperature operation in order to achieve a high quality signal and/or low noise measurements. By contrast, a germanium-silicon (GeSi) single-photon avalanche diode (SPAD) that can operate in room temperature has been reported (Na, N., Lu, YC., Liu, Y H. et al., “Room temperature operation of germanium-silicon single-photon avalanche diode,” Nature (2024), incorporated herein by reference), which paves the way towards many new applications. A GeSi SPAD may detect SWIR single-photons at room temperature without requiring cryogenic conditions as conventional SWIR single-photon detectors do. Further, a GeSi SPAD may be compatible with complementary metal-oxide-semiconductor (CMOS) fabrication processes.

Accordingly, the systems and methods herein may utilize GeSi SPADs, which may have high sensitivity to light and may overcome challenges related to the limited penetration depth of light in the visible and near-infrared range.

The present disclosure describes an optical sensing apparatus that utilizes SPAD photodetectors such as GeSi SPAD photodetectors to perform detection of chemicals such as glucose in the body. The optical sensing apparatus may use time-resolved spectroscopy with one or more light sources in selected Near-Infrared (NIR) and/or SWIR ranges, where light interactions with other molecules (e.g., non-target molecules that are not of interest such as water, hemoglobin, muscle, lipid, etc.) are low. Moreover, SPAD devices can be used for direct time-of-flight (dToF) measurements, which provide time-resolved information related to the tissues. The time-resolved information may help to identify properties of the blood vessels where glucose resides. SPAD devices, such as GeSi SPAD devices, have suitable properties such as being broadband, highly sensitive, large-area, and cost-effective at SWIR spectrum, and can provide a less noisy detection through time-resolved, space-resolved, and/or wavelength-resolved measurements. In addition, since the glucose concentrations are obtained directly from the photon counts in the time-domain, e.g., from dToF histograms, at one or several wavelengths including SWIR, less or no extravagant or computationally-heavy data processing or big data-driven algorithms would be required. Further, using time-domain techniques, the present disclosure may reduce or avoid the signal offset caused by the light entering and exiting the tissue. Accordingly, the systems and methods disclosed herein may overcome challenges related to interference of other molecules, signal offset, and limited penetration depths of NIR wavelengths, as discussed previously.

The present disclosure further describes a machine-learning model to analyze the detected data for glucose detection. A machine-learning model allows multiple types of data (e.g., spectral, temporal, spatial etc.) associated with how SWIR light interacts with various user characteristics (e.g., skin color, gender, age, etc.) to be used for determining target chemical information (e.g., glucose concentrations, glucose concentrations over time, changes in blood flow), which could further improve the accuracy of the detection. The machine-learning model may comprise one or more neural networks. The present disclosure further describes utilizing a plurality of channels that may improve the accuracy of the machine-learning model despite any nonlinearity and imperfection that may exist in measurements. The plurality of channels may comprise spectral channels (which may provide spectrally resolved properties), and/or temporal channels (which may provide time-resolved properties), and/or spatial channels (which may provide distance-resolved properties).

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

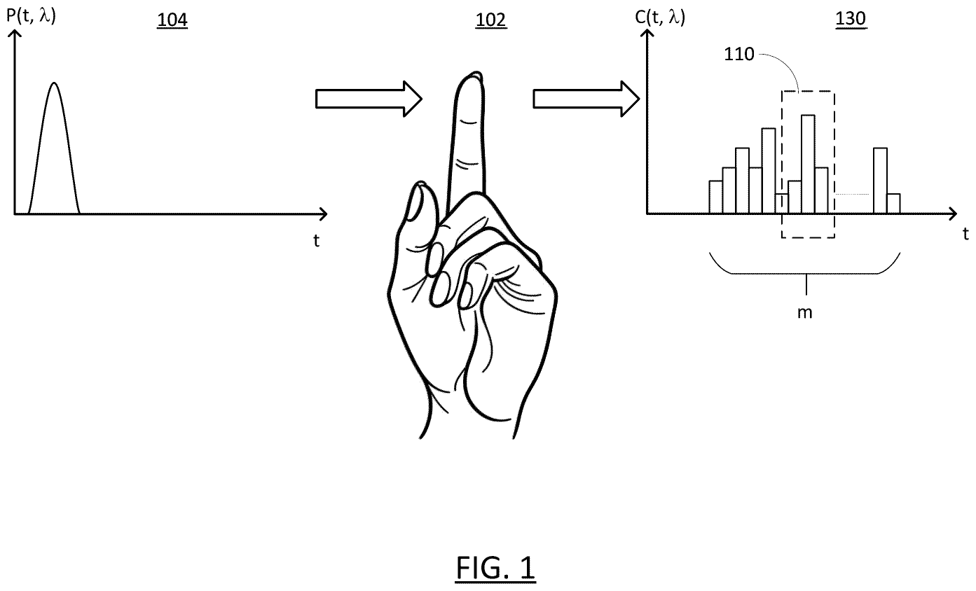

FIG. 1 shows an example of a system 100 that illustrates using SPAD photodetectors to perform glucose detection. The SPAD photodetectors may be GeSi SPAD photodetectors or other SPAD photodetectors formed using another material or material compound. The system 100 may be a mobile device, a wearable device (e.g., a ring, an armband, a wristband, earbuds, a neckband, etc.), or any other suitable device/system. The system 100 includes one or more transmitters, one or more receivers, and one or more processors (not shown).

The transmitter is configured to emit light pulses 104 towards a measurement target, such as biological tissues 102 (e.g., a finger as shown in FIG. 1). In some implementations, the light pulses may have one or more wavelengths. The system may comprise one or more transmitters to emit light pulses at a reference wavelength. The reference wavelength may be selected from a wavelength range where glucose has low absorbency and the detected intensity of light at that wavelength remains stable and independent of glucose concentration. The system may comprise one or more transmitters to emit light pulses at one or more signal wavelengths. In general, the one or more signal wavelengths are selected such that these wavelengths interact more with glucose molecules and less with other molecules (e.g., hemoglobin, water, muscle, lipid, etc.) in the tissue. The one or more reference and signal wavelengths may comprise wavelengths in the NIR range, the SWIR range, the mid-infrared (MIR) range, or a combination of ranges. For example, the NIR range may be a wavelength range from about 780 nm to about 1000 nm, or any similar wavelength range as defined by a particular application. The SWIR range may be a wavelength range from about 1000 nm to about 3000 nm, or any similar wavelength range as defined by a particular application. The MIR range may be a wavelength range from about 3000 nm to about 8000 nm, or any similar wavelength range as defined by a particular application.

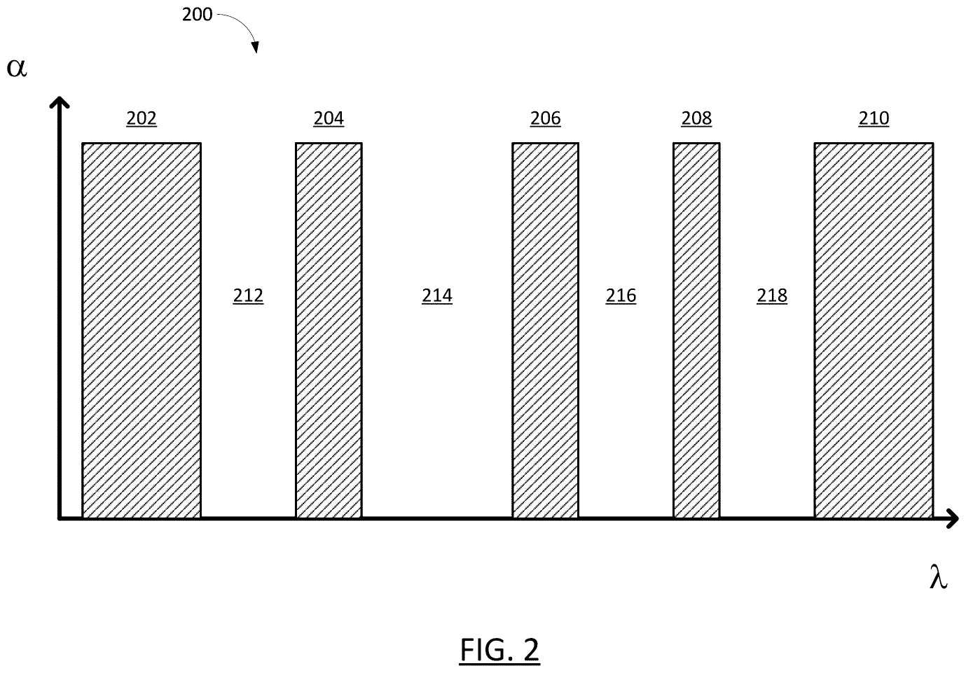

Selecting the one or more wavelengths may comprise determining wavelengths where glucose has a high absorbance (e.g., over a first specific absorbance threshold) and where other molecules have low absorbance (e.g., under a second first specific absorbance threshold). As an example, FIG. 2 shows a relationship graph 200 between wavelengths (λ) and absorption coefficients (α). The wavelength bands (or wavelength ranges; these two terms are used interchangeably throughout the disclosure) 202, 204, 206, 208, and 210 comprise non-target wavelengths, generally to be avoided for glucose detection. For example, the wavelength band 202 may be from 700 nm to 1000 nm, where the absorption coefficients for hemoglobin are high in this band. As another example, the wavelength bands 204, 208, and 210 may be from 1400 nm to 1500 nm, from 1900 nm to 2000 nm, and from 2250 nm to 2500 nm, respectively, where the absorption coefficients for water are high in these bands. As another example, the wavelength bands 206 and 210 may be from 1700 nm to 1800 nm and from 2250 nm to 2500 nm, respectively, where the absorption coefficients for lipid are high in these bands. On the other hand, the wavelength bands 212, 214, 216, and 218 may comprise target wavelengths, which are desirable candidates as light in these wavelength bands may interact more with glucose molecules and less with water, lipid, and hemoglobin. For example, the transmitter may be configured to emit light pulses in the wavelength bands from 1000 nm to 1400 nm (e.g., wavelength band 212), from 1500 nm to 1700 nm (e.g., wavelength band 214), from 1800 nm to 1900 nm (e.g., wavelength band 216), and/or from 2000 nm to 2250 nm (e.g., wavelength band 218).

In some implementations, the system may comprise a multiplexer and a demultiplexer. Pulses with one or more wavelengths may be multiplexed in the time domain, such that time-resolved signals for one or more wavelength channels at the receiver end can be obtained and analyzed with temporal demultiplexing.

In some implementations, the system may comprise a multiplexer and a demultiplexer. In some implementations, the pulses with one or more wavelengths may be multiplexed in the space domain, such that time-resolved signals for one or more wavelength channels at the receiver end can be obtained and analyzed with spatial demultiplexing.

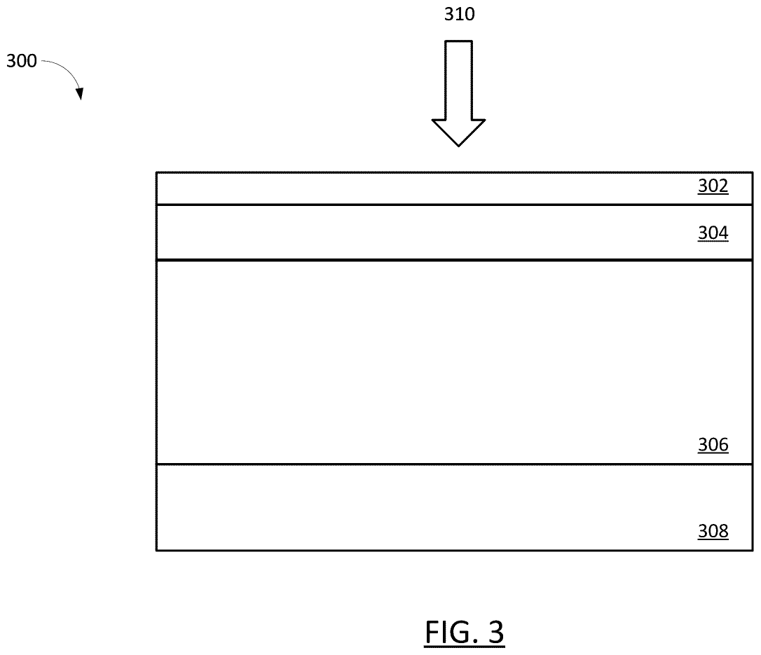

In general, biological tissues comprise different layers. FIG. 3 illustrates a simplified representation of a biological tissue 300 having different layers, including skin layer 302 (stratum corneum), epidermis layer 304, dermis layer 306 (interstitial fluids), and hypodermis layer 308 (subcutaneous tissue), with a typical thickness of 0.03 mm, 0.1 mm, 1.5 mm, and 5 mm, respectively. In general, blood vessels are in the dermis layer 306 and the hypodermis layer 308, where the glucose molecules can be detected. However, as the light pulses 310 enter the biological tissues, the light pulses interact with all layers of the biological tissues, including the skin layer 302 and the epidermis layer 304. To separate the glucose detection signals from others, as explained below, the system 100 uses direct time-of-flight (dToF) techniques to identify particular time durations where the light pulses have interacted with glucose molecules.

In some examples, the transmitters may emit light pulses towards the biological tissue 300, the biological tissue 300 may absorb at least some of the emitted light, and the receivers may detect the transmitted light that has not been absorbed by the biological tissue 300. The transmitters may be positioned on one side of the biological tissue (e.g., finger 102 shown in FIG. 1), and the receivers may be positioned on another side of the biological tissue.

In some examples, the transmitters may emit light pulses towards the biological tissue, the biological tissue 300 may absorb, scatter, and/or reflect at least some of the emitted light, and the receivers may detect the reflected light. The transmitters may be positioned on the same side of the biological tissue (e.g., finger, wrist or ear) as the receivers, for example.

When light passes through different tissues, for example, skin layer 302, epidermis layer 304, dermis layer 306, and/or hypodermis layer 308, the light may be scattered. The light may be scattered in different directions due to the heterogenous nature of tissue in the different skin layers.

Referring back to FIG. 1, the receiver includes one or more SPADs that are configured to receive light pulses having one or more wavelengths that have interacted with biological tissues over a plurality of time cycles, and convert the light pulses into a plurality of electrical signals. The SPADs may be GeSi SPADs. An example of GeSi SPAD can be found in the article “Room temperature operation of germanium-silicon single-photon avalanche diode” (Na, N., Lu, YC., Liu, Y H. et al., Nature (2024), incorporated herein by reference). In some implementations, the GeSi SPAD may be modified to include tin (Sn) to increase the operation wavelength. In some implementations, the SPADs may be arranged as a one-dimensional (1D) array or a two-dimensional (2D) array to further increase the detection area.

The receiver may be configured to receive light pulses having one or more wavelengths that have been reflected by biological tissue. By comparing how much light has been received by the receiver compared to how much light was emitted by the one or more transmitters, the system may determine how much light was absorbed and scattered by the biological tissue.

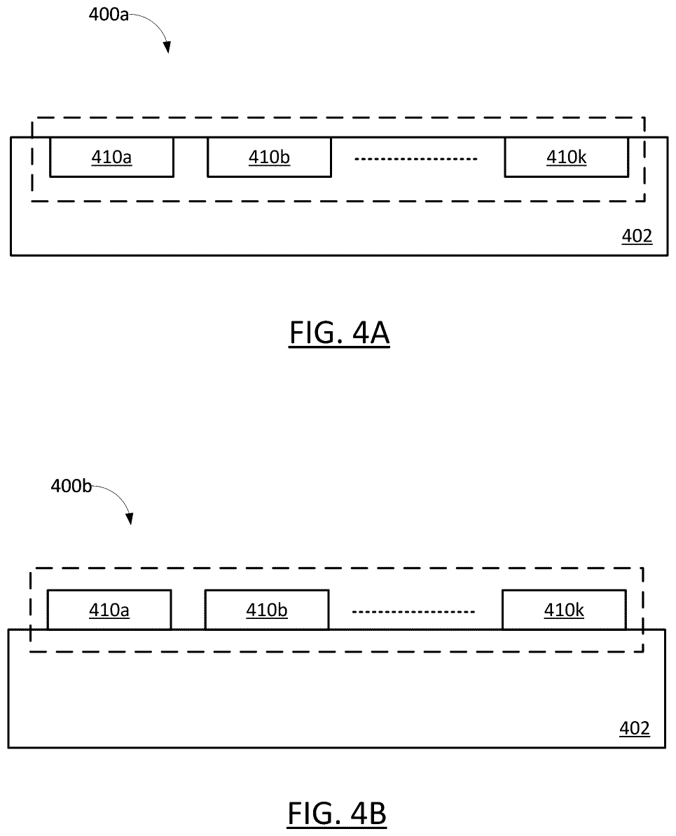

FIGS. 4A and 4B show examples of an SPAD photodetector 400a/400b. The SPAD photodetector 400a/400b includes a substrate 402 (e.g., silicon or another substrate material) and k pixels 410a-410k along a direction, where each of the k pixels 410a-410k may be an SPAD having germanium (Ge) (or germanium containing tin) absorption regions. In some embodiments, SPAD photodetector 400a/400b may be a GeSi SPAD photodetector. In some embodiments, one or more of the k pixels 410a-410k may be GeSi SPADs. GeSi SPADs may be compatible with CMOS fabrication processes. SPAD photodetector 400a/400b may be able to detect light on a single photon scale at room temperature.

Referring to FIG. 4A, in some implementations, the k pixels 410a-410k may be partially or fully embedded in the substrate 402 (e.g., epitaxially grown in an etched trench in the substrate 402). For example, the k pixels 410a-410k may be epitaxially grown using molecular beam epitaxy, chemical vapor deposition, or other known methods of epitaxial growth.

Referring to FIG. 4B, in some implementations, the k pixels 410a-410k may be formed as a mesa on a surface of the substrate 402 (e.g., epitaxially grown over the surface of the substrate 402, followed by an etch to form the pixels). For example, the k pixels 410a-410k may be epitaxially grown on the surface of substrate 402 using molecular beam epitaxy, chemical vapor deposition, or other known methods of epitaxial growth. Then, the surface may be etched using wet etching, dry etching, or other known etching techniques.

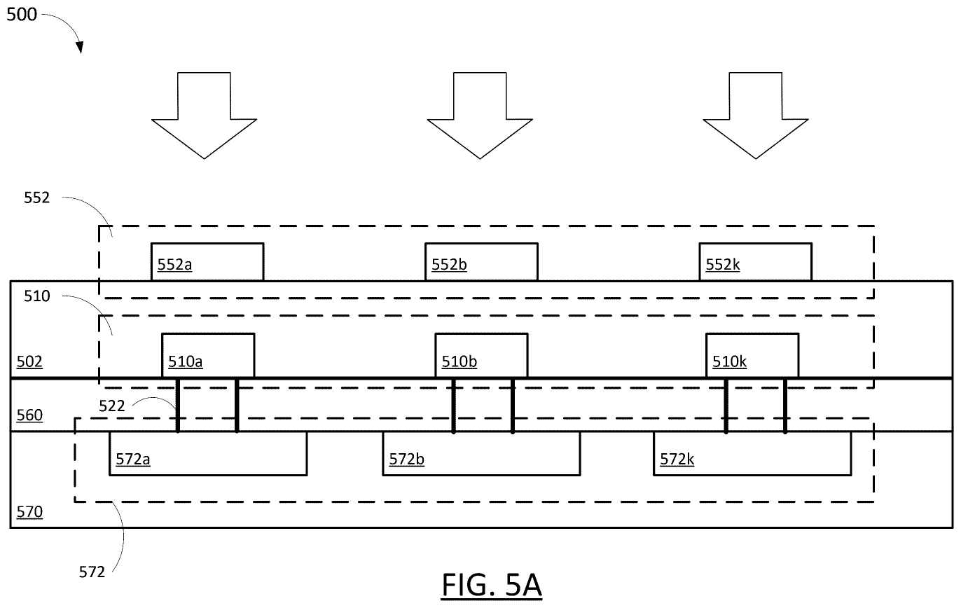

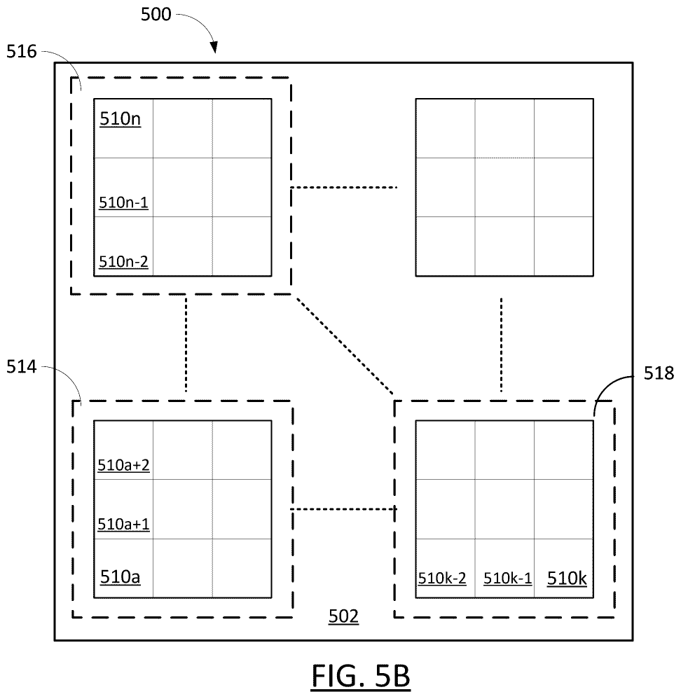

FIG. 5A shows an example of a 2D SPAD array photodetector 500. The photodetector 500 includes a first substrate 502 (e.g., silicon substrate) having k pixels 510a-510k (e.g., k pixels 410a-410k described in reference to FIGS. 4A and 4B) along a direction that is directly or flipped bonded to a second substrate 570 (e.g., a silicon substrate) having circuitry 572a-572k (collectively, circuitry 572). The bonding may be done at chip-level or wafer-level. In some embodiments, one or more of k pixels 510a-510k may be SPADs having germanium absorption regions. In some embodiments, one or more of k pixels 510a-510k may be SPADs having germanium containing tin absorption regions. The bonding interface 560 may include a dielectric material (e.g., oxide), a metallic material (e.g., copper), or a mix between a dielectric material and a metallic material. In some implementations, the circuitry 572 may include k individual circuitry that each is electrically coupled to a corresponding pixel 510 via wires 522. The circuitry 572 may be readout circuitry that is configured to configured to process the plurality of electrical signals from the SPAD to generate a plurality of digital electrical signals that can be further processed. For example, the circuitry 572 may include a time-to-digital converter that provides a count of photons entering the SPAD over time, where a histogram (e.g., histogram 130 of FIG. 1, described in more detail below) can then be constructed over multiple pulses.

In some implementations, the photodetector 500 may include an optical structure 552. The optical structure 552 may be configured to pass target wavelengths to the pixels 510. The optical structure 552 may include one or more layers of structures that focuses, directs, filters, passes, or otherwise manipulates an optical signal that enters the k pixels 510. In some implementations, the optical structure 552 may include k optical structures 552a-552k that each is optically coupled to a corresponding pixel 510 (pixel 510a, 510b, and 510k, respectively). For example, an optical structure 552a may be a band pass filter that is implemented using a meta-surface, a Fabry-Perot interferometer, or an absorption material. As another example, an optical structure 552a may be a combination of a band pass filter and an optical lens, where the band pass filter may be implemented using a meta-surface, a Fabry-Perot interferometer, or an absorption material, and the optical lens may be implemented using a meta-surface or a convex lens (e.g., silicon lens, silicon nitride lens, or polymer lens).

In some embodiments, the optical structure 552 may be configured to block non-target wavelengths. The optical structures 552a-552k may be configured to block one or more non-target wavelengths in the NIR range, the SWIR range, and/or the MIR range. For example, the optical structures 552a-552k may be configured to block one or more of the wavelength ranges of 700 nm to 1000 nm (wavelength band 202 of FIG. 2), 1400 to 1500 nm (wavelength band 204 of FIG. 2), 1900 nm to 2000 nm (wavelength band 208 of FIG. 2), and/or 2250 to 2500 nm (wavelength band 210 of FIG. 2).

In some implementations, different optical structures may be configured to pass a different wavelength range of light. For example, the optical structures 552a, 552b, and 552k may be configured to pass wavelength bands 212, 214, and 216, respectively, comprising target wavelengths. In some embodiments, the optical structures 552a-552k may be configured to pass wavelengths of light where glucose may have a high absorbance, but other molecules may have a low absorbance. In some embodiments, the optical structures 552a-552k may be configured to pass light in the NIR range, and/or the SWIR range, and/or the MIR range. In some embodiments, the optical structures 552a-552k may be configured to pass one or more of the wavelength ranges of 1000 nm to 1400 nm (wavelength band 212 of FIG. 2), 1500 nm to 1700 nm (wavelength band 214 of FIG. 2), 1800 nm to 1900 nm (wavelength band 216 of FIG. 2), and/or 2000 nm to 2250 nm (wavelength band 218 of FIG. 2).

FIG. 5B illustrates an example of a top view of a 2D SPAD array photodetector 500 having a two-dimensional array of k×n pixels. In some implementations, pixels may be logically binned by the associated bandpass filters (e.g., optical structure 552). The bandpass filters may be configured to filter one or more wavelengths. In some embodiments, a bandpass filter that passes a selected wavelength range may be formed over a grouping of pixels (e.g., pixel group 514, pixel group 516, or pixel group 518), and the grouping of pixels may be configured to detect light in that selected wavelength range. As an example, a bandpass filter that passes the wavelength band 212 may be formed over a 3×3 pixel group 514, such that the pixel group 514 is configured to detect light in the wavelength band 212. Moreover, a bandpass filter that passes the wavelength band 214 may be formed over a 3×3 pixel group 516, such that the pixel group 516 is configured to detect light in the wavelength band 214. A bandpass filter that passes the wavelength band 216 may be formed over a 3×3 pixel group 518, such that the pixel group 518 is configured to detect light in the wavelength band 216. While FIG. 5B shows pixel group 514, pixel group 516, and pixel group 518 as 3×3 pixel groupings, examples of the disclosure may include any arrangement of pixel groupings, including more than 3×3 or less than 3×3. In some examples, the pixel groupings may be arranged as rows and columns of pixels. The number of rows and the number of columns of pixels in a pixel grouping may be different. For example, a pixel group may comprise a 3×4 pixel grouping, a 3×5 pixel grouping, etc.

In some aspects, the circuitry 572 may be configured to separately control each grouping of pixels, which may simplify the overall control of the photodetector. In some implementations, the circuitry 572 may be configured to dynamically control how the pixel groups operate over time in order to increase the flexibility of the device.

In some embodiments, different bandpass filters may pass wavelength bands to different pixels. For example, a bandpass filter that passes the wavelength band 212 may be formed over pixels including 510a, 510n−2, and 510k−2, such that the pixel group is configured to detect light in the wavelength band 212. Moreover, a bandpass filter that passes the wavelength band 214 may be formed over pixels including 510a+1, 510n−1, and 510k−1, such that the pixel group is configured to detect light in the wavelength band 214. A bandpass filter that passes the wavelength band 216 may be formed over pixels 510a+2, 510n, and 510k, such that the pixel group is configured to detect light in the wavelength band 216. Here, such arrangement may allow the photodetector 500 to have a better detectable resolution for different light spectra. In some implementations, the circuitry 572 may be configured to dynamically control how the pixel groups operate over time in order to increase the flexibility of the device as described above. For example, if three pulses in the wavelength bands 212, 214, and 216 arrive at the SPAD array photodetector 500 at three different points of time, the circuitry 572 may turn on a corresponding pixel group at corresponding time while turning off other pixel groups in order to reduce crosstalk noises and to save power.

Referring back to FIG. 1, the one or more processors are configured to identify, for each of the plurality of pulse cycles, particular time slot(s) 110 representing a time duration that each of the light pulses has interacted with glucose molecules. FIG. 1 shows a histogram C(t, λ) 130 for multiple time cycles (e.g., pulse cycle), where the histogram can be sliced into m time slots. The particular time slot(s) 110 where a light pulse has interacted with glucose molecules can be identified if information associated with the optical path of the system (e.g., the length of the biological tissues, and/or the diffusion length of the biological tissues, and/or the effective index of the biological tissues, and/or a distance between the system and the biological tissues, and/or whether the measurement is transmissive or reflective, and/or whether the light pulses have transmitted through or reflected from the biological tissues and/or an absorption coefficient of the biological tissues and/or a scattering coefficient of the biological tissues) is known. For example, assuming a post-transmission histogram distribution of 1.5 ns and a temporal resolution of 50 ps, 20 time slots can be sliced. The one or more processors can identify the particular time slot(s) 110 from the 20 time slots based on the location and the thickness of the blood vessels, which can be predetermined or dynamically adjusted. In some embodiments, the particular time slot(s) may be determined based on a timing jitter associated with one or more SPADs (e.g., substantially equal to, or longer than, or shorter than the timing jitter).

The one or more processors are configured to determine, based on a subset of the plurality of digital electrical signals that correspond to the particular time slots, one or more characteristics (e.g., glucose concentration, glucose concentration variation over time, etc.) of glucose molecules associated with the biological tissues. In some implementations, the one or more processors may determine the one or more characteristics of the glucose molecules using a machine-learning model.

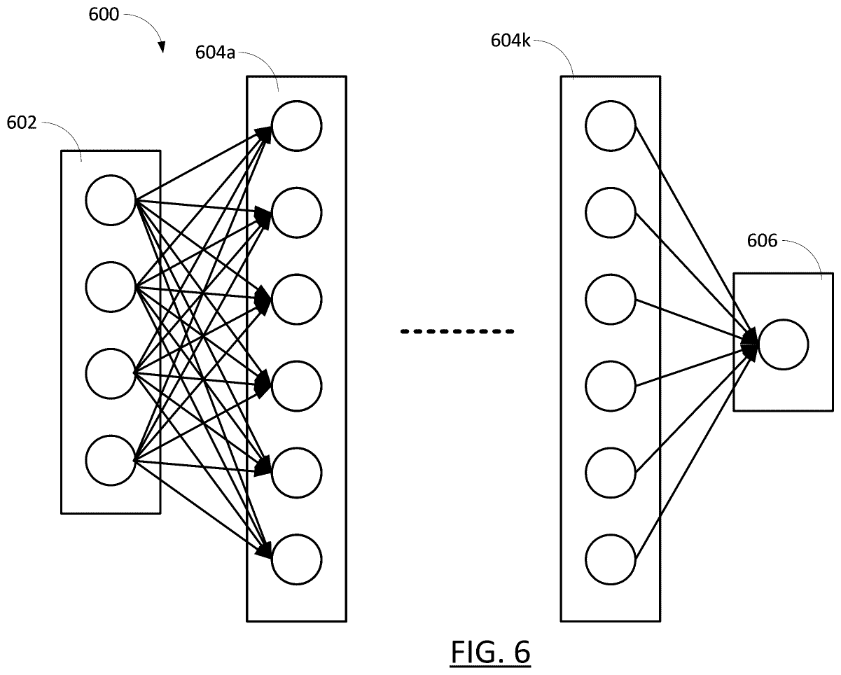

FIG. 6 shows an example neural network model 600 that has been trained to output one or more characteristics of chemicals such as measured glucose molecules. In some implementations, the one or more characteristics of the measured glucose molecules may comprise glucose concentration, glucose concentration over time, or a combination thereof. The neural network model 600 may include an input layer 602, one or more k hidden layers 604a-604k, and an output layer 606. The input layer 602 receives preprocessed data represented as numerical vectors. In some embodiments, the preprocessed data may have been preprocessed by one or more processors of a non-invasive glucose monitoring system (e.g., a system similar to system 100 of FIG. 1). The preprocessed data can include spectrally-resolved measurements (e.g., measurement outputs from optical pulses with one or more wavelengths), temporally-resolved measurements (e.g., measurement outputs from the particular time slots in 110), spatially-resolved measurements (e.g., measurement outputs from the particular pixel locations in photodetector 500), measurements over multiple pulses of the same wavelengths/time slots/pixel locations, and/or other relevant factors, such as various user characteristics relevant to how light (e.g., NIR light, SWIR light or MIR light) interacts with biological tissues (e.g., skin color, gender, age etc.). Each of the one or more k hidden layers 604a-604k contains neurons trained and implemented by known techniques (e.g., backpropagation for weight assignments, etc.). The output layer 606 is configured to produce an output that represents the one or more characteristics (e.g., glucose concentration, glucose concentration variation over time, etc.) of glucose molecules associated with the biological tissues.

Using a neural network model or equivalent machine-learning model further improves the accuracy of the measurements, as the model can be trained based on larger population samples, such that random anomalies from the measurements can be included over time. The neural network 600 may further comprise a bias layer. Neural network 600 may be trained using experimental glucose measurements obtained from capillary, venous, and/or serum samples. While FIG. 6 illustrates example neural network comprising 4 channels in input layer 602, it should be understood that neural network 600 may have any number of channels in input layer 602. Similarly, while FIG. 6 illustrates example neural network comprising 1 channel in output layer 606, it should be understood that neural network 600 may have any number of channels in output layer 606.

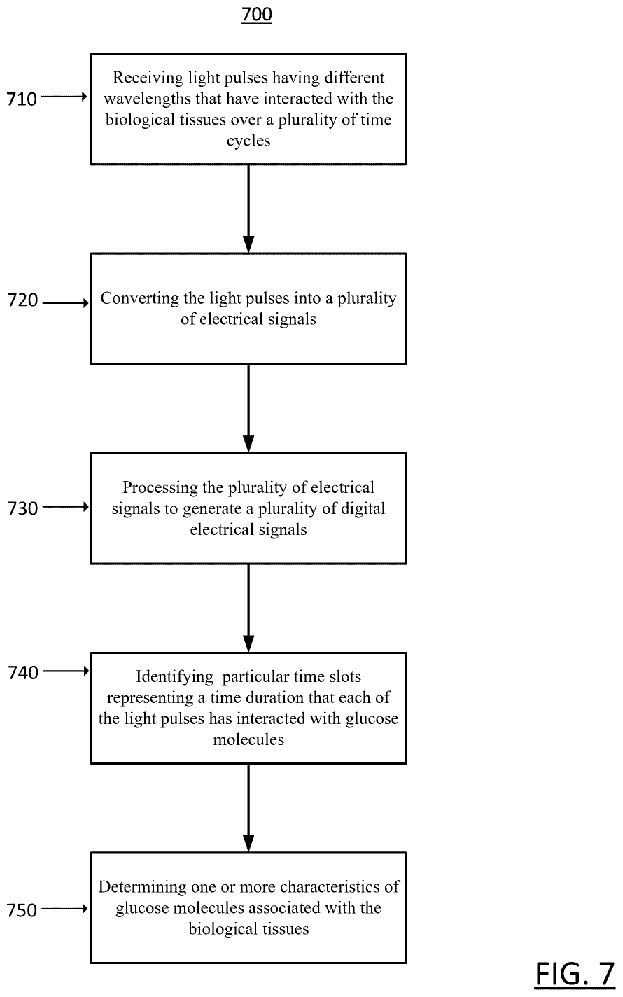

FIG. 7 shows an example flow 700 for detecting glucose in biological tissues using an optical sensing apparatus, where the flow 700 can be implemented by the system 100 or the system 800, as an example. The flow comprises receiving, by one or more single-photon avalanche diodes, light pulses having one or more wavelengths that have interacted with the biological tissues over a plurality of time cycles; converting, by the one or more single-photon avalanche diodes, the light pulses into a plurality of electrical signals, where each of the one or more single-photon avalanche diodes comprises an absorption region formed on a substrate, where the absorption region comprises germanium or germanium containing tin, and where the substrate comprises silicon; processing, by circuitry, the plurality of electrical signals to generate a plurality of digital electrical signals; identifying, by one or more processors and for each of the plurality of time cycles, particular time slots representing a time duration that each of the light pulses has interacted with glucose molecules; and determining, by the one or more processors and based on a subset of the plurality of digital electrical signals that correspond to the particular time slots, one or more characteristics of glucose molecules associated with the biological tissues.

At step 710, one or more SPADs may receive light pulses. The one or more SPADs may be GeSi SPADs. In some embodiments, the GeSi SPADs may be configured to detect pulses of light on a photon scale, such as a single photon scale. The light pulses may comprise one or more (target) wavelengths of light. In some embodiments, the one or more wavelengths of light may be in the NIR range, and/or the SWIR range, and/or the MIR range. In some embodiments, the one or more wavelengths of light may be selected such that the target molecule (e.g., glucose) may have a high absorbance of the target wavelengths, while other (non-target) molecules (e.g., hemoglobin, water, and/or lipids) have a low absorbance of the non-target wavelengths. In some embodiments, the plurality of different target wavelengths may comprise wavelengths in one or more of the 1000 nm to 1400 nm range, the 1500 nm to 1700 nm range, the 1800 nm to 1900 nm range, and/or the 2000 nm to 2250 nm range. The light pulses may have interacted with biological tissues over a plurality of time cycles. In some embodiments, the light pulses may have interacted with one or more layers of biological tissue. The one or more layers of biological tissue may comprise one or more of a skin layer (e.g., stratum corneum), an epidermis layer, a dermis layer (which may comprise interstitial fluids), and/or a hypodermis layer (which may comprise subcutaneous tissue).

Step 720 of flow 700 may comprise converting the received light pulses into a plurality of electrical signals. The received light pulses may be converted into a plurality of electrical signals by the one or more SPADs.

Step 730 of flow 700 may comprise processing the plurality of electrical signals. Processing the plurality of electrical signals may comprise generating a plurality of digital electrical signals. Circuitry of an SPAD photodetector (e.g., circuitry 572 of SPAD photodetector 500 of FIG. 5) may process the plurality of electrical signals to generate a plurality of digital signals. In some embodiments, the circuitry of the SPAD photodetector may comprise readout circuitry. For example, the circuitry may include a time-to-digital converter that provides a plurality of digital signals representing one or more counts of photons entering an SPAD over time.

Step 740 of flow 700 may comprise identifying one or more particular time slots. In some embodiments, the one or more particular times slots represent a time duration that each of the light pulses has interacted with glucose molecules. Identification of the one or more particular time slots may be based on known information associated with an optical path of the system. In some embodiments, the known information associated with an optical path of the system comprises one or more of a length of the biological tissues, a diffusion length of the biological tissues, an effective index of the biological tissues, an absorption coefficient of the biological tissues, a scattering coefficient of the biological tissues and/or a distance between the system and the biological tissues. In some embodiments, identifying the one or more particular time slots may be done experimentally and may comprise utilizing dToF techniques. Time slots may be measured based on a post-transmission histogram distribution and a temporal resolution. The one or more time slots may be identified by one or more processors (e.g., the one or more processors of system 100 of FIG. 1). Identifying the one or more particular time slots where light pulses have interacted with glucose may improve accuracies by enabling separation of glucose detection signals from other signals.

Step 750 of flow 700 may comprise determining one or more characteristics of glucose molecules associated with the biological tissues. The one or more characteristics of glucose molecules may comprise glucose concentration, glucose contraction variation over time, or a combination thereof. The one or more characteristics of glucose may be determined by one or more processors (e.g., the one or more processors of system 100 in FIG. 1). The one or more processors may comprise one or more machine-learning models (e.g., neural network model 600 of FIG. 6). The one or more processors may determine the one or more characteristics of glucose molecules based on one or more of the identified time slots, one or more characteristics associated with interactions between the signal wavelength and glucose molecules, one or more characteristics associated with interactions between the reference wavelength and glucose molecules, input intensities of the light pulses, and/or output intensities of the light pulses.

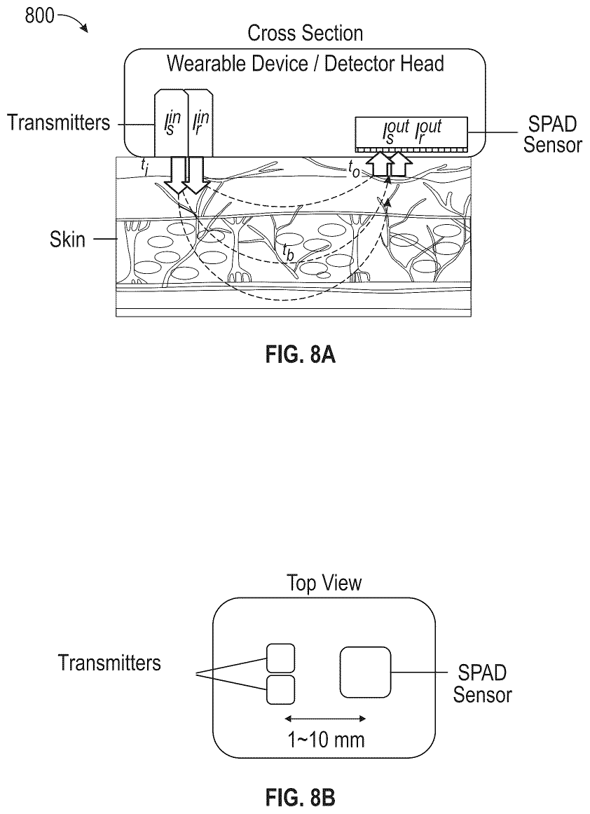

FIGS. 8A and 8B show a cross-section view and a top view of another example system 800 that uses SPAD photodetectors to perform chemical (e.g., glucose) detection. The system 800 includes two transmitters, where a first transmitter emits light pulses (e.g., sub-nanosecond pulse width with a predetermined pulse cycle) at a reference wavelength (e.g., a wavelength in the SWIR range), and a second transmitter emits light pulses (e.g., sub-nanosecond pulse width with a predetermined pulse cycle) at a signal wavelength (e.g., a different wavelength in the SWIR range). The reference wavelength may be chosen from a wavelength range where glucose has a low absorbency, such that the detected reference light intensity remains stable and independent of glucose concentration. The signal wavelength may be selected from a range of wavelengths where glucose may have a high absorbance and other molecules may have a low absorbance. For example, the signal wavelength may be selected from one or more of the ranges of 1000 nm to 1400 nm, 1500 nm to 1700 nm, 1800 nm to 1900 nm, and/or 2000 nm to 2250 nm.

The system 800 further includes one or more single-photon avalanche diodes configured to receive light pulses with the signal wavelength and the reference wavelength, and convert the light pulses into a plurality of electrical signals. The one or more SPADs may comprise one or more GeSi SPADs. The one or more single-photon avalanche diodes may be implemented using the SPAD photodetector 400a/400b and/or the 2D SPAD array photodetector 500 described above. In general, the transmitters are configured to illuminate the target skin tissue, and the one or more single-photon avalanche diodes collect the reflected/scattered photons from the target skin tissue as a function of flight time. As illustrated in FIG. 8B, a spacing between the SPAD photodetector and the at least two transmitters may be between about 1 mm and 10 mm, according to some embodiments.

The system 800 further includes one or more processors and/or equivalent circuitry configured to determine, based on the plurality of electrical signals, a time duration that each of the light pulses has interacted with glucose molecules. The light pulses may interact with all layers of the biological tissues in addition to the layers that may comprise blood vessels and blood glucose. To separate the glucose detection signals from other signals, system 800 may utilize dToF techniques to determine the time duration that each of the light pulses has interacted with glucose molecules. In some embodiments, the duration that each of the light pulses has interacted with glucose molecules may be determined based on known information associated with an optical path of the system 800. In some embodiments, the known information associated with an optical path of the system comprises one or more of a length of the biological tissues, a diffusion length of the biological tissues, an effective index of the biological tissues, an absorption coefficient of the biological tissues, a scattering coefficient of the biological tissues, and/or a distance between the system 800 and the biological tissues.

In some implementations, the one or more processors may be configured to determine one or more characteristics (e.g., concentration) of glucose molecules associated with the biological tissues based on (i) the time duration, (ii) one or more characteristics associated with interactions between the signal wavelength and glucose molecules, (iii) one or more characteristics associated with interactions between the reference wavelength and glucose molecules, (iv) input intensities of the light pulses, (v) output intensities of the light pulses, or a combination thereof. In some embodiments, the one or more processors may comprise one or more neural networks. The one or more neural networks may comprise one or more of an input layer, one or more hidden layers, a bias, a sigmoid function, and/or an output layer. The one or more neural networks may be trained using experimental glucose measurements obtained from capillary, venous, and/or serum samples. The one or more neural networks may comprise a plurality of channels, which may improve the accuracy of the machine-learning model despite nonlinearities and imperfections that may exist in the training experiments. The plurality of channels may comprise spectral channels (which may provide spectrally resolved properties), and/or temporal channels (which may provide time-resolved properties), and or spatial channels (which may provide distance-resolved properties).

Referring to FIG. 8A as an example, Iₛⁱⁿ and Iₛ^{out} indicate the input and output short pulse (e.g., sub-nanosecond pulse width with a predetermined pulse cycle, or longer pulse width as long as the depth resolution is sufficient) SWIR light intensities, respectively, at the signal wavelength λₛ. Iᵣⁱⁿ and Iᵣ^{out} indicate the input and output short pulse SWIR light intensities, respectively, at the reference wavelength λᵣ. The output light intensities can be expressed as:

$${I}_{s}^{out} = {I}_{s}^{i n} {t}_{i ( s )} {t}_{b ( s )} {t}_{o ( s )} {e}^{- {α}_{s} ( n ) \cdot L} ; \\ and \qquad ( 1 )$$

$${I}_{r}^{out} = {I}_{r}^{i n} {t}_{i ( r )} {t}_{b ( r )} {t}_{o ( r )} {e}^{- {α}_{r} ( n ) \cdot L} , \qquad ( 2 )$$

where tᵢ, t_{b}, tₒ are the optical losses for light entering the skin, transmitting inside the skin body, and exiting the skin, respectively, where αₛ(n), αᵣ(n) are the skin absorption coefficients at λₛ and λᵣ, respectively, as a function of glucose concentration n, and where L is the transmission length in the skin body.

In some implementations, L can be represented by L=ν·τ, where ν is the speed of light in the biological tissues at the signal wavelength (e.g., speed of light in free space divided by the effective index of the biological tissues). In some embodiments, the effective index of the biological tissues at the signal wavelength may be approximately equal to the effective index of the biological tissues at the reference wavelength. Moreover, τ is the transmission time that the light pulses have interacted with glucose molecules, where τ can be measured by dToF measurements during operations. In some implementations, L can be represented by L=√(2Dτ), where D is the diffusion coefficient and τ is the transmission time.

By comparing the two output intensities, the ratio of the output intensities can be expressed as:

$$\frac{{I}_{s}^{out}}{{I}_{r}^{ou}} = \frac{{I}_{s}^{i n} {t}_{i ( s )} {t}_{b ( s )} {t}_{o ( s )} {e}^{- {α}_{s} ( n ) \cdot L}}{{I}_{r}^{i n} {t}_{i ( r )} {t}_{b ( r )} {t}_{o ( r )} {e}^{- {α}_{r} ( n ) \cdot L}} . \qquad ( 3 )$$

In some implementations, the signal and reference wavelengths λₛ and λᵣ can be chosen to be close to each other (e.g., 1350 nm and 1400 nm) such that tᵢ₍ₛ₎t_{b(s)}tₒ₍ₛ₎≈tᵢ₍ᵣ₎t_{b(r)}tₒ₍ᵣ₎, the speed ν is a constant (or approximately the same) across the two wavelengths and

$$\frac{{I}_{s}^{out}}{{I}_{r}^{ou}} = \frac{{I}_{s}^{i n} {e}^{{α}_{s} ( n ) \cdot L}}{{I}_{r}^{i n} {e}^{{α}_{r} ( n ) \cdot L}} .$$

Representation (3) can then be further simplified as:

$$ln ( \frac{{I}_{s}^{out}}{{I}_{r}^{out}} ) = ln ( \frac{{I}_{s}^{i n}}{{I}_{r}^{i n}} ) + [ {α}_{r} ( n ) - {α}_{s} ( n ) ] \cdot v τ ; \\ and \qquad ( 4 )$$

$$ln ( \frac{{I}_{s}^{out}}{{I}_{r}^{out}} ) - ln ( \frac{{I}_{s}^{i n}}{{I}_{r}^{i n}} ) = [ {ω}_{r} - {ω}_{s} ] n \cdot v τ , \qquad ( 5 )$$

where ωₛ and ωᵣ are the absorption cross-sections at wavelengths λₛ and λᵣ, respectively.

Based on Representation (5), the glucose concentration can be determined as:

$$n = \frac{1}{[ {ω}_{s} - {ω}_{r} ] \cdot v} \cdot \frac{[ ln ( \frac{{I}_{s}^{i n}}{{I}_{r}^{i n}} ) - ln ( \frac{{I}_{s}^{out}}{{I}_{r}^{out}} ) ]}{τ} , \qquad ( 6 )$$

where the coefficient

$$\frac{1}{[ {ω}_{s} - {ω}_{r} ] \cdot v}$$

can further be expressed as C, and where n can be expressed as:

$$n = C \cdot \frac{[ ln ( \frac{{I}_{s}^{i n}}{{I}_{r}^{i n}} ) - ln ( \frac{{I}_{s}^{out}}{{I}_{r}^{out}} ) ]}{τ} . \qquad ( 7 )$$

Note that Iₛⁱⁿ, Iₛ^{out}, Iᵣⁱⁿ, Iᵣ^{out}, and τ can be measured during operations. In some implementations, the one or more processors may determine the time duration that each of the light pulses has interacted with glucose molecules (τ) by identifying, for each of the plurality of time cycles, particular time slots representing the time duration that each of the light pulses has interacted with glucose molecules. In some implementations, the one or more processors may identify the particular time slots representing the time duration by identifying the particular time slots based on a histogram comparison between the light pulses at the signal wavelength and the light pulses at the reference wavelength.

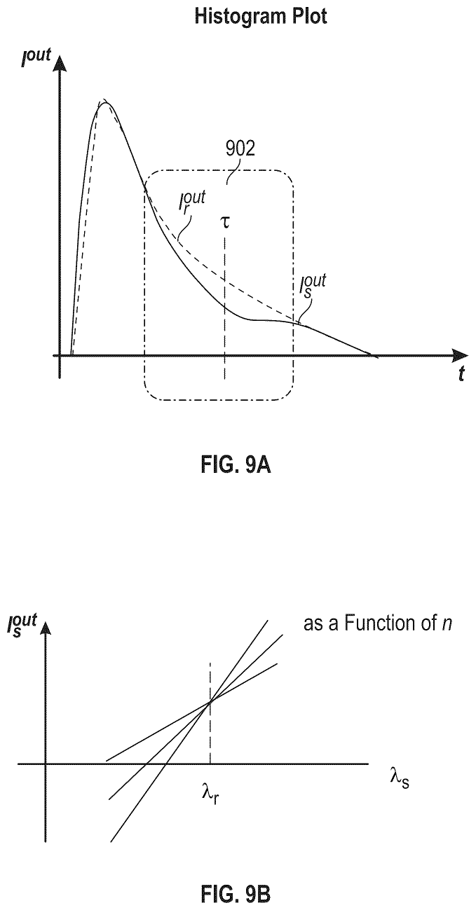

Referring to FIG. 9A as an example, the output signal and reference light intensities can be measured and transformed into photon counts in the time-domain histograms. The difference between the measured output signal and the reference light intensities may be determined from the time-domain histogram. Since the two wavelengths react with glucose molecules differently (e.g., different absorption coefficients, and/or scattering coefficients, and/or effective index), the one or more processors may identify the time window 902 where the output intensities differ between the two wavelengths by a predetermined threshold (e.g., 10% difference). τ is the transmission time that the light pulses have interacted with glucose molecules. In some embodiments, τ may be measured by dToF measurements during operations. In some embodiments, τ may be calculated based on known information associated with an optical path of the system.

Referring to FIG. 9B as an example, in some implementations, the reference wavelength may be chosen as a wavelength where Iᵣ^{out} is substantially independent from the glucose concentration n, such that the reference light intensity is stable regardless of varying glucose concentration. For example, the reference wavelength may be chosen such that [ωᵣ−ωₛ]n is equal to about −ωₛn.

In some implementations, the one or more processors may determine the time duration that each of the light pulses has interacted with glucose molecules by determining an average time duration over multiple time durations that the light pulses have interacted with glucose molecules. Referring back to FIG. 9A, the average time duration that the light pulses has interacted with glucose molecules may be determined by averaging the time window where the output intensities differ between the two wavelengths by a predetermined threshold over multiple light pulses.

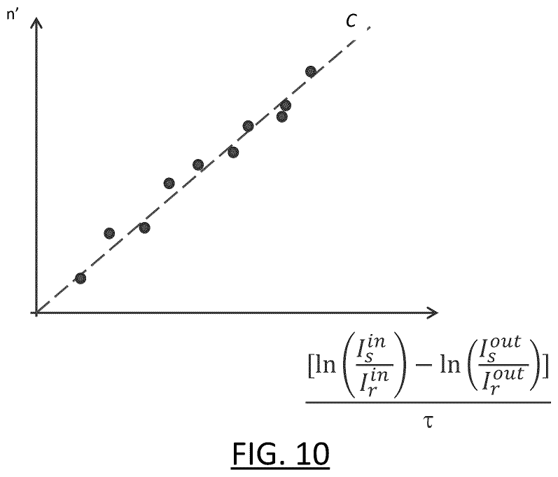

In some implementations, the absorption cross-sections ωₛ and ωᵣ and the speed ν may be derived from existing values in known literature, and C can be predetermined accordingly. In some other implementations, C may be predetermined experimentally. Referring to FIG. 10 as an example, the ground truth glucose concentrations n′ for a user may be obtained by a traditional invasive glucose monitor (e.g., CGM) over several conditions and time periods, and the corresponding Iₛⁱⁿ, Iₛ^{out}, Iᵣⁱⁿ, Iᵣ^{out}, and τ can be measured non-invasively for the user in parallel. The coefficient C can be fitted through a linear regression or machine-learning algorithm, which may more accurately reflect how user characteristics (e.g., skin color, gender, age, etc.) affect how SWIR light interacts with biological tissues. This correlation data may be used for predicting glucose concentration through non-invasive glucose monitoring.

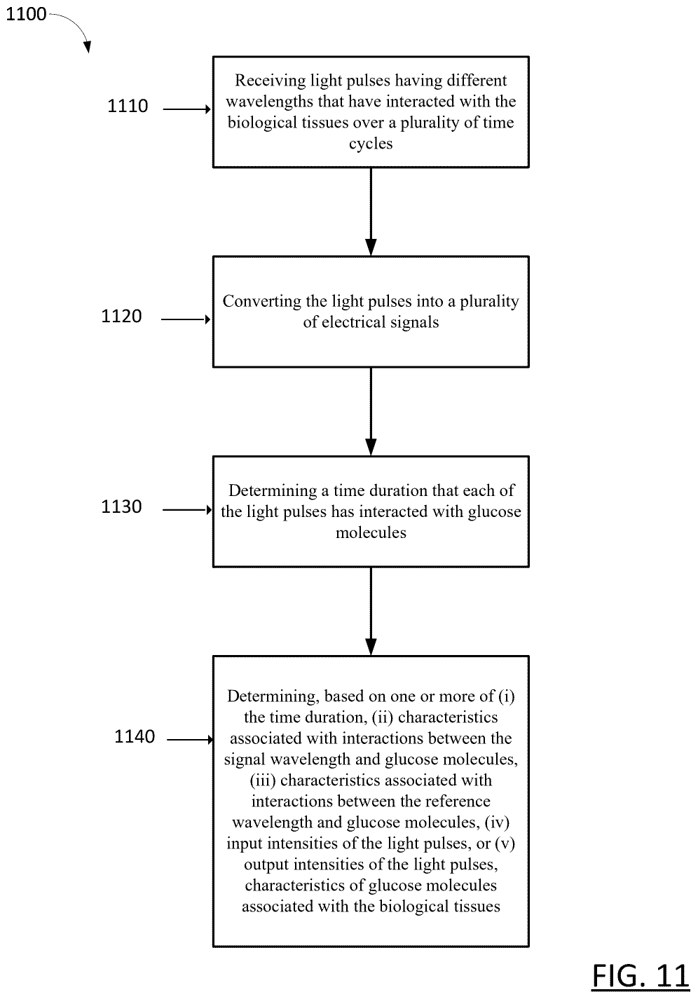

FIG. 11 shows an example flow 1100 for detecting glucose in biological tissues using an optical sensing apparatus, where the flow 1100 can be implemented by any of the disclosed systems (system 100, system 800, as examples). The flow 1100 includes receiving, by the one or more single-photon avalanche diodes, light pulses having at least two wavelengths that have interacted with biological tissues over a plurality of time cycles, where the at least two wavelengths include a signal wavelength and a reference wavelength; converting, by the one or more single-photon avalanche diodes, the light pulses into a plurality of electrical signals, where each of the one or more single-photon avalanche diodes comprises an absorption region formed on a substrate, where the absorption region comprises germanium or germanium containing tin, and where the substrate comprises silicon; determining, based on the plurality of electrical signals, a time duration that each of the light pulses has interacted with glucose molecules; and determining one or more characteristics of glucose molecules associated with the biological tissues, based on (i) the time duration, and/or (ii) one or more characteristics associated with interactions between the signal wavelength and glucose molecules, and/or (iii) one or more characteristics associated with interactions between the reference wavelength and glucose molecules, and/or (iv) input intensities of the light pulses, and/or (v) output intensities of the light pulses, and/or a combination thereof.

Step 1110 of flow 1100 comprises receiving light pulses. The light pulses may be received by one or more SPADs. The one or more SPADs may be GeSi SPADs. In some embodiments, the SPADs may be configured to detect pulses of light on a photon scale (e.g., single photon scale). The light pulses may comprise at least two wavelengths of light. In some embodiments, the at least two wavelengths of light may be in the NIR range, the SWIR range, and/or the MIR range. In some embodiments, the at least two wavelengths may comprise a reference wavelength and one or more signal wavelengths. In some embodiments, the reference wavelength may be selected such that glucose has a low absorbency of that wavelength. In some embodiments, the one or more signal wavelengths of light may be selected such that target chemical (e.g., glucose) may have a high absorbance of the target wavelengths, while other molecules (e.g., hemoglobin, water, and/or lipids) have a low absorbance of those wavelengths. In some embodiments, the one or more signal wavelengths may comprise wavelengths in one or more of the 1000 nm to 1400 nm range, the 1500 nm to 1700 nm range, the 1800 nm to 1900 nm range, and/or the 2000 nm to 2250 nm range. The light pulses may have interacted with biological tissues over a plurality of time cycles. In some embodiments, the light pulses may have interacted with one or more layers of biological tissue. The one or more layers of biological tissue may comprise one or more of a skin layer (e.g., stratum corneum), an epidermis layer, a dermis layer (which may comprise interstitial fluids), and/or a hypodermis layer (which may comprise subcutaneous tissue).

Step 1120 of flow 1100 may comprise converting the received light pulses into a plurality of electrical signals. The received light pulses may be converted into a plurality of electrical signals by the one or more SPADs.

Step 1130 of flow 1100 may comprise determining, based on the plurality of electrical signals, a time duration that each of the light pulses has interacted with the target chemical (e.g., glucose molecules). In some embodiments, the time duration may be determined experimentally based on a histogram comparison between the detected intensities of the light pulses at the signal wavelength and the detected intensities of the light pulses at the reference wavelength. In some embodiments, the time duration may be calculated based on known information associated with an optical path of the light pulses.

Step 1140 of flow 1100 may comprise determining characteristics of glucose molecules associated with biological tissues based on a plurality of factors. The plurality of factors may comprise one or more of the determined time duration, one or more characteristics associated with interactions between the signal wavelength and glucose molecules, one or more characteristics associated with interactions between the reference wavelength and glucose molecules, a plurality of input intensities of the light pulses, and/or a plurality output intensities of the light pulses. The one or more characteristics of glucose molecules may comprise glucose concentration, glucose contraction variation over time, or both. The one or more characteristics of glucose may be determined by one or more processors (e.g., the one or more processors of system 100 in FIG. 1). The one or more processors may comprise one or more machine-learning models (e.g., neural network model 600 of FIG. 6).

Examples of the disclosure are based on the sensing mechanisms and architectures described above, and the targeted analytes are not limited to glucose. As long as the target chemical can be identified through appropriately chosen wavelengths, one or more properties of such target chemical can be derived using the sensing mechanisms and architectures described above.

Unless otherwise specified, as used herein, the terms “photodetector”, “optical sensor”, “optical sensing apparatus”, or other similar terms can include a device that has been designed and/or operated as a photodiode (PD), an avalanche photodiode (APD), a single-photon avalanche diode (SPAD), or a locked-in PD (LIPD).

As used herein, the terms such as “first”, “second”, “third”, “fourth” and “fifth” describe various elements, components, regions, layers and/or sections, these elements, components, regions, layers and/or sections should not be limited by these terms. These terms may be only used to distinguish one element, component, region, layer or section from another. The terms such as “first”, “second”, “third”, “fourth”, and “fifth” when used herein do not imply a sequence or order unless clearly indicated by the context. The terms “photo-detecting”, “photo-sensing”, “light-detecting”, “light-sensing”, and any other similar terms can be used interchangeably.

Spatial descriptions, such as “above”, “over”, “under”, “top”, and “bottom” and so forth, are indicated with respect to the orientation shown in the figures unless otherwise specified. It should be understood that the spatial descriptions used herein are for purposes of illustration only, and that practical implementations of the structures described herein can be spatially arranged in any orientation or manner, provided that the merits of embodiments of this disclosure are not deviated by such arrangement.

As used herein and not otherwise defined, the terms “substantially” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

While the concepts have been described by way of examples and in terms of embodiments, it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

1. An optical sensing apparatus for glucose detection, comprising:
one or more single-photon avalanche diodes configured to:
receive light pulses having one or more wavelengths that have interacted with biological tissues over a plurality of time cycles; and
convert the light pulses into a plurality of electrical signals,
wherein each of the one or more single-photon avalanche diodes comprises:
an absorption region formed on a substrate, wherein the absorption region comprises germanium or germanium containing tin, and wherein the substrate comprises silicon; and
circuitry configured to process the plurality of electrical signals to generate a plurality of digital electrical signals; and
one or more processors configured to:
identify, for each of the plurality of time cycles, particular time slots representing a time duration that each of the light pulses has interacted with glucose molecules; and
determine, based on a subset of the plurality of digital electrical signals that correspond to the particular time slots, one or more characteristics of glucose molecules associated with the biological tissues.

2. The optical sensing apparatus of claim 1, wherein the one or more single-photon avalanche diodes are arranged in a one-dimensional array or a two-dimensional array.

3. The optical sensing apparatus of claim 2, wherein determining the one or more characteristics of the glucose molecules associated with the biological tissues further comprises determining, based on a subset of the plurality of digital electrical signals that correspond to specific pixel locations in the one-dimensional array or the two-dimensional array, the one or more characteristics of glucose molecules associated with the biological tissues.

4. The optical sensing apparatus of claim 1, wherein the one or more single-photon avalanche diodes comprise one or more wavelength filters arranged on the absorption regions for passing the one or more wavelengths of the light pulses.

5. The optical sensing apparatus of claim 1, wherein each of the particular time slots is determined based on a timing jitter associated with the one or more single-photon avalanche diodes.

6. The optical sensing apparatus of claim 1, wherein the circuitry comprises a time-to-digital converter.

7. The optical sensing apparatus of claim 1, wherein identifying the particular time slots further comprises identifying the particular time slots based on one or more of an effective index associated with the biological tissues, an absorption coefficient associated with the biological tissues, or a scattering coefficient associated with the biological tissues.

8. The optical sensing apparatus of claim 7, wherein identifying the particular time slots further comprises identifying the particular time slots based on determining that the light pulses have transmitted through the biological tissues.

9. The optical sensing apparatus of claim 7, wherein identifying the particular time slots further comprises identifying the particular time slots based on determining that the light pulses have reflected from the biological tissues.

10. The optical sensing apparatus of claim 1, wherein determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

11. The optical sensing apparatus of claim 1, wherein the light pulses have wavelengths in two or more of (i) a range between 1000 nm to 1400 nm, (ii) a range between 1500 nm to 1700 nm, (iii) a range between 1800 nm to 1900 nm, or (iv) a range between 2000 nm to 2250 nm.

12. A method for detecting glucose in biological tissues using an optical sensing apparatus, the method comprising:
receiving, by one or more single-photon avalanche diodes, light pulses having one or more wavelengths that have interacted with the biological tissues over a plurality of time cycles;
converting, by the one or more single-photon avalanche diodes, the light pulses into a plurality of electrical signals, wherein each of the one or more single-photon avalanche diodes comprises an absorption region formed on a substrate, wherein the absorption region comprises germanium or germanium containing tin, and wherein the substrate comprises silicon;
processing, by circuitry, the plurality of electrical signals to generate a plurality of digital electrical signals;
identifying, by one or more processors and for each of the plurality of time cycles, particular time slots representing a time duration that each of the light pulses has interacted with glucose molecules; and
determining, by the one or more processors and based on a subset of the plurality of digital electrical signals that correspond to the particular time slots, one or more characteristics of glucose molecules associated with the biological tissues.

13. The method of claim 12, wherein the one or more single-photon avalanche diodes are arranged in a one-dimensional array or a two-dimensional array.

14. The method of claim 13, wherein determining the one or more characteristics of the glucose molecules associated with the biological tissues further comprises determining, based on a subset of the plurality of digital electrical signals that correspond to specific pixel locations in the one-dimensional array or the two-dimensional array, one or more characteristics of glucose molecules associated with the biological tissues.

15. The method of claim 12, wherein each of the particular time slots is determined based on a timing jitter associated with the one or more single-photon avalanche diodes.

16. The method of claim 12, wherein identifying the particular time slots further comprises identifying the particular time slots based on one or more of an effective index associated with the biological tissues, an absorption coefficient associated with the biological tissues, or a scattering coefficient associated with the biological tissues.

17. The method of claim 16, wherein identifying the particular time slots further comprises identifying the particular time slots based on determining that the light pulses have transmitted through the biological tissues.

18. The method of claim 16, wherein identifying the particular time slots further comprises identifying the particular time slots based on determining that the light pulses have reflected from the biological tissues.

19. The method of claim 12, wherein determining the one or more characteristics of the glucose molecules associated with the biological tissues further comprises determining the one or more characteristics of the glucose molecules using a neural network model, and wherein input data for the neural network model includes wavelength information of the light pulses or the subset of the plurality of digital electrical signals from different temporal or spatial domains.

20. An optical sensing apparatus for glucose detection, comprising:
one or more single-photon avalanche diodes configured to:
receive light pulses that have interacted with biological tissues over a plurality of time cycles; and
convert the light pulses into a plurality of electrical signals,
wherein each of the one or more single-photon avalanche diodes comprises an absorption region formed on a substrate, wherein the absorption region comprises germanium or germanium containing tin, and wherein the substrate comprises silicon;
circuitry configured to process the plurality of electrical signals to generate a plurality of digital electrical signals; and
one or more processors configured to:
identify, for each of the plurality of time cycles, particular time slots representing a time duration that each of the light pulses has interacted with glucose molecules; and
determine, using a machine-learning model and based on a subset of the plurality of digital electrical signals that correspond to the particular time slots, one or more characteristics of glucose molecules associated with the biological tissues.
